(19) United States
(12) Patent Application Publication
(10) Pub. No.: US 2003/0224026 A1
(43) Pub. Date: Dec. 4, 2003

(54) Dermatological or cosmetic composition which includes aromatic nitroxide compounds and their use

(76) Inventors: Lucedio Greci, Felino (IT); Elisabetta Damiani, Cagli (IT)

Correspondence Address: MCCARTER & ENGLISH LLP, CITYPLACE I, 185 ASYLUM STREET, HARTFORD, CT, 06103, US

(21) Appl. No.: 10/368,782

(22) Filed: Feb. 19, 2003

(30) Foreign Application Priority Data
Feb. 19, 2002 (IT) MI2002A000332

(51) Int. Cl. A61K 7/42; A61K 7/00; A61K 31/404

(52) U.S. Cl. 424/401; 514/418; 424/59

(57) ABSTRACT

The present invention refers to a composition which includes cyclic nitroxide compounds. In particular, the present invention refers to a dermatological and/or cosmetic composition which includes mono- and bis-cyclic nitroxides. Furthermore, the present invention concerns with the use of mono- and bis-cyclic nitroxides for the preparation of a composition for the dermatological and/or cosmetic treatment of skin, and methods of treating the skin using such compounds.

### PRIOR APPLICATION

This application claims priority to the Italian Patent No. MI2002A000332 filed in Italy on Feb. 19, 2002, which is incorporated herein by reference in its entirety.

### FIELD OF THE INVENTION

The present invention relates to a dermatological and/or cosmetic composition in which cyclic nitroxide compounds are included. In particular, the present invention relates to a dermatological and/or cosmetic composition which includes mono- and bis-cyclic nitroxide compounds. In addition, the present invention relates to the use of mono- and bis-cyclic nitroxide compounds for the preparation of a composition for the dermatological and/or cosmetic treatment of skin.

### BACKGROUND

It is known that among the most common forms of cancer which affects the human being, skin cancer is without doubt one of the most widespread. In particular, in the last twenty years, the number of human beings afflicted with skin cancer has increased remarkably. Part of this increase is due to the fact, that for the typical person, the amount of time exposed to solar radiation, both natural and artificial, has increased. Overexposure of skin to solar radiation has contributed to the increasing use of sunscreens. Some researchers claim that the sunscreens may themselves be subjected to photolytic reactions induced by light, with the consequent formation of free radicals harmful to a healthy skin. Furthermore, solar radiation has become less filtered due to the decrease in the ozone layer.

The facts mentioned above could also be responsible for the increase in many pathologies to which certain types of skin are subjected to.

In the scientific field, it is believed that the ultraviolet component of solar radiation (UV rays) plays a primary role in inducing skin tumours since these UV rays directly attack cells, damaging their DNA.

The use of nitroxide radicals as antioxidant agents for combating oxidative stress caused by free radicals is well known. Furthermore, the use of nitroxide radicals as antioxidant agents for limiting the damage caused by exposure to UVA rays in different biological systems is known.

Among the defensive measures adopted to photoprotect skin from diseases induced by overexposure to solar radiation, the use of sun filters, or sunscreens, is probably the measure most commonly adopted.

The presence on the market of certain kinds of sun creams which contain a various number of antioxidant agents and sun filters is known. The antioxidant agents used belong to various categories of compounds such as vitamin E and ascorbic acid.

Among the antioxidant compounds which have been proposed for this purpose, there is a first category of cyclic mono-nitroxide radicals, such as, for example 2,2,6,6-tetramethylpiperidine-1-oxyl (TEMPO) and 2,2,6,6-tetramethylpiperidine-3-hydroxy-1-oxyl (TEMPOL). These are both cyclic aliphatic nitroxides.

A second category of compounds are cyclic bis-nitroxide radicals, for example [bis(2,2,6,6-tetramethylpiperidine-1-oxyl-4-yl)sebacate] (aliphatic piperidine bis-nitroxide), for simplicity referred to by the abbreviation TDN.

Cosmetic creams containing sun filters or cosmetic creams which are available on the market contain a plurality of antioxidant agents and coformulants. However, the use of antioxidant agents in a cream containing sun filters or in a cream for dermatological and/or cosmetic purposes has various inconveniences or disadvantages.

A first disadvantage is due to the fact that some compounds, among which certain antioxidant agents cannot be excluded, once applied on the skin, could themselves be subjected to degradation due to sun rays with the consequent formation of harmful free radicals. The free radicals formed can themselves be harmful for the skin. Furthermore, the free radicals formed can chemically react with other radicals present in the sun cream. Also, the free radicals formed can chemically react with other compounds/coformulants present in the sun cream.

Therefore, it is increasingly necessary or desirable to have at one's disposal compounds to filter the sun's rays which have been tested, and which have a controlled action and are of reliable efficiency.

Another disadvantage of existing products is due to the fact that when the number of compounds, or antioxidant agents used in a cosmetic cream is increased, it becomes increasingly difficult to prepare a product with characteristics of high stability in time.

Yet another disadvantage of existing products is due to the fact that on increasing the number of compounds, or antioxidant agents used in a cosmetic cream, it becomes increasingly difficult to prepare a product with characteristics of high compatibility in relation to the various skin types on which it will be applied.

In fact, it may happen that the topical application of a cream containing a sun filter, or a cosmetic cream, induces certain allergic reactions due to the actual specific chemical composition of the product employed.

Furthermore, another disadvantage is the fact that on increasing the number of active antioxidant agents used in the preparation of a cream containing a sun filter, or a cosmetic cream, the costs of the final product increases.

Therefore, it is necessary to be able to have at one's disposal new antioxidant agents which do not have the inconveniences of the commonly accepted products or method.

In particular, it is necessary to be able to have at one's disposal antioxidant agents that possess high efficacy and chemical stability with time.

Therefore, one object of the present invention is that of having at hand new antioxidant agents which can be employed in the preparation of cosmetic and/or dermatological compositions aimed at an always more vast population.

Another object of the present invention is to make a cosmetic and/or dermatological composition which includes these antioxidant agents and substances acceptable from the dermatological and cosmetic point of view.

A further object of the present invention is to propose the use of these antioxidant agents for the preparation of a dermatological and/or cosmetic composition for the treatment of skin.

These objects and others which will appear clear during the detailed description which will follow, have been reached by the Applicant, who found it useful to select certain nitroxide radicals as antioxidant agents.

### BRIEF DESCRIPTION OF THE DRAWINGS

Further technical characteristics and the advantages of the discovery will be illustrated in greater detail with reference to the attached figures, given only for indicative purposes, and therefore, not restrictive, in which:

FIG. 1 shows a graph with time on the x axis and the consumption of oxygen on the y axis measured with an oxygraph. The graph shows the effects of the nitroxides tested on the consumption of oxygen (8 independent experiments) during the oxidation of 2.5 mM PC liposomes (PC=phosphatidylcholine) in 5 mM phosphate buffer saline 0.1 mM EDTA, pH 7.4, at 37° C. Oxidation was induced by injecting at point A, 25 μl of Bu^{t}OOH 7.3 M and injecting at point B 25 μl of cytochrome C (1 mg protein/ml). Point C refers to the addition of 5 μM of the following compounds (if not specified otherwise):

in trial (a) the compound with formula (I) described below and in claim 1 at a concentration of 8 μM in which R=(X) and (X) contains R1=methylenic C4 alkyl residue (DC4) was tested: in trial (b) the compound with formula (I) in which R=(X) and (X) contains R1=C4 alkyl residue (DC4) was tested; in trial (c), the compound with formula (I) in which R=methylenic C4 alkyl residue (MC4) was tested; in trial (d), the compound with formula (I) in which R=C8 alkyl residue (MC8) was tested; in trial (e), the compound with formula (I) in which R=(X) and (X) contains R1-C8 methylenic alkyl residue (DC8) was tested; in trial (f) [bis(2,2,6,6-tetramethylplperidine-1-oxyl-4-yl)sebacate)] (aliphatic piperidine bis-nitroxide), abbreviated for simplicity to (TDN); in trial (g) acetonitrile (control); in trial (h) TEMPO.

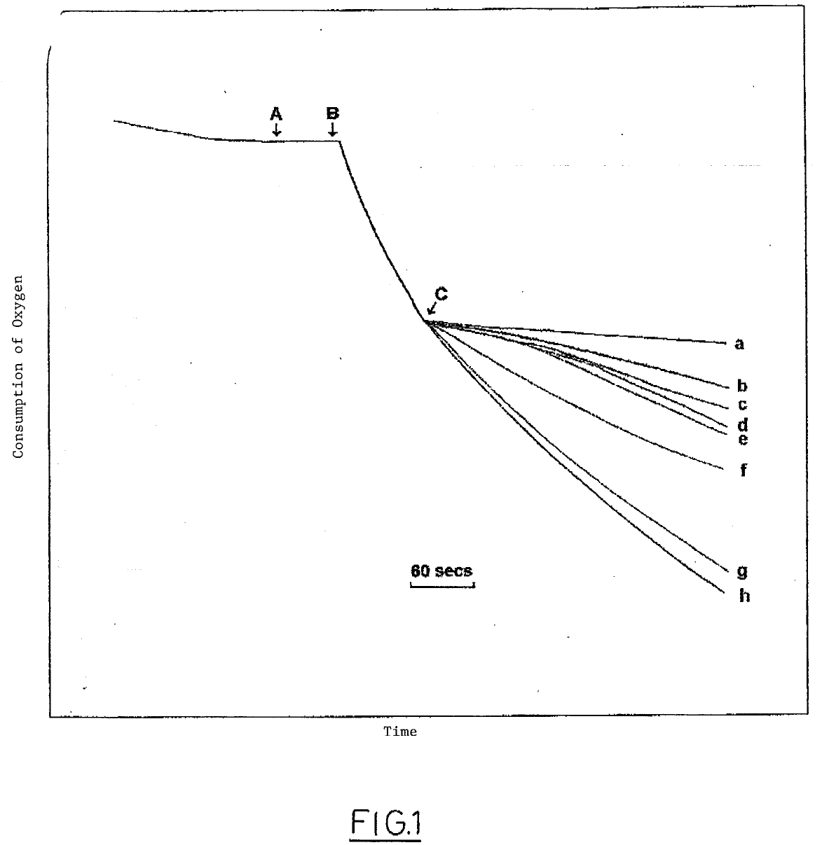

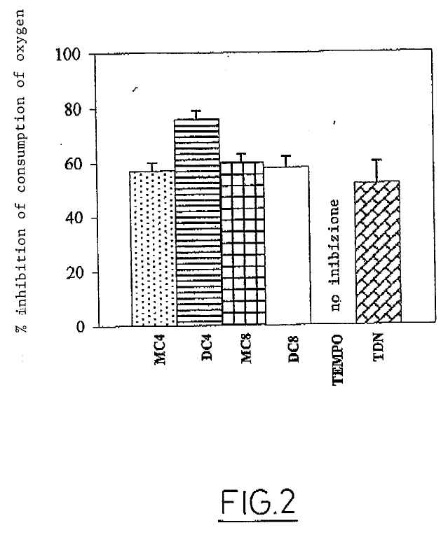

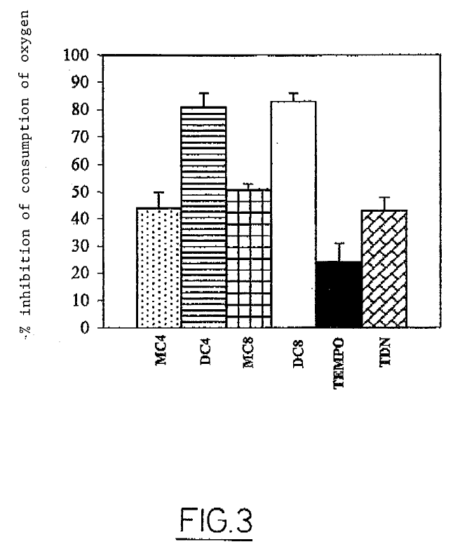

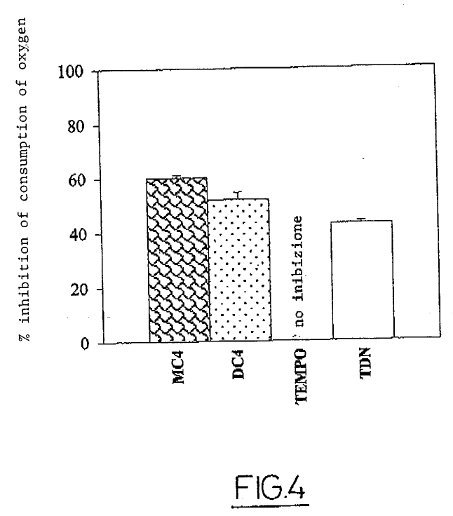

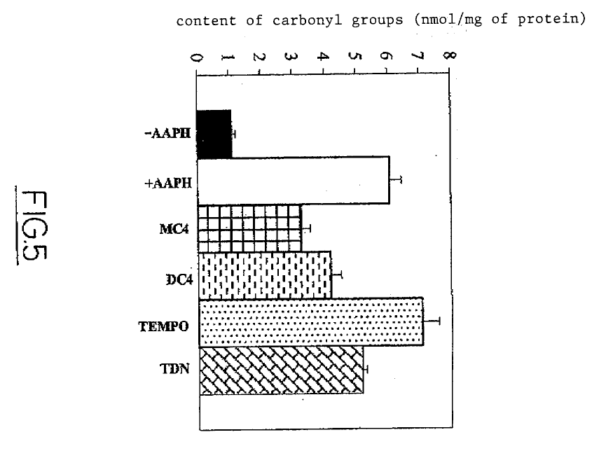

FIG. 2 shows a histogram with the compounds reported below on the x axis and the % inhibition of the consumption of oxygen on the y axis. The compounds are indicated with the following abbreviations: MC4, the compound with formula (I) in which R=C4 alkyl residue; DC4, the compound with formula (I) in which R=(X) and (X) contains R1=C4 methylenic alkyl residue; MC8, the compound with formula (I) in which R=C8 alkyl residue; DC8, the compound with formula (I) in which R=(X) and (X) contains R1=C8 methylenic alkyl residue; TEMPO and TDN. With reference to FIG. 2, the percentage inhibition of the consumption of oxygen in PC unilamellar liposome (2.4 mM) in 5 mM phosphate buffer saline, 0.1 mM EDTA, pH 7.4 at 37° C. to which 5 μM nitroxide compounds was added are reported. The values were calculated from measurements similar to those shown in FIG. 1.

FIG. 3 shows a histogram with the compounds reported below on the x axis and the % inhibition of the consumption of oxygen on the y axis. The compounds are indicated with the following abbreviations: MC4, DC4, MC8, DC8, TEMPO and TDN as defined in FIG. 2. With reference to FIG. 3, the percentage inhibition of the consumption of oxygen is obtained from measurements carried out on an oxygraph. The system studied consists of linolenic acid micelles (3 mM) in 30 mM Tris-HCl bufer, pH 8.5, at 37° C. to which 5 μM of nitroxides are added. The values were calculated from measurements similar to those reported in FIG. 1.

FIG. 4 shows a histogram with the compounds reported below on the x axis and the % inhibition of oxygen consumption on the ⁻y axis. The compounds are indicated with the abbreviations: MC4, DC4, TEMPO and TDN as defined for FIG. 2. With reference to FIG. 4, the percentage inhibition of the consumption of oxygen was obtained from measurements carried out on an oxygraph. The system studied consists of multilamellar PC liposomes which have nitroxides incorporated inside them (221M) in 5 mM phosphate buffer saline, 0.1 mM EDTA, pH 7.4 at 37° C. The values were calculated from measurements similar to those shown in FIG. 1.

FIG. 5 shows a histogram with the compounds reported below on the x axis and the content of carbonyl groups (nmol/mg of protein) on the y axis. The compounds are indicated with the abbreviations: MC4, DC4, MC4, MC8, TEMPO and TDN as defined for FIG. 2. With reference to FIG. 5, the system studied consists of a sample of 3 mg/ml of bovine serum albumin in 50 mM phosphate buffer pH 7.4. The sample is incubated with 5 mM of [2,2′-azobis(2-amidinopropane)dihydrochloride] (azo-initiator) at 50° C. for 60 minutes, in the presence of absence of the addition of 70 μM nitroxides. Subsequently, the modification of the oxidative process was evaluated.

### DETAILED DESCRIPTION

In a first embodiment of the present invention is a cosmetic or dermatological composition comprising aromatic cyclic nitroxide compounds with the following general formula (I),

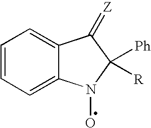

in which:

Z represents an oxygen or a NR₂ group with R₂ representing an alkyl, alkoxy, aryl or phenyl group; R represents a C1-C18 alkyl residue, a phenyl or a substituted benzene residue, a benzylic or substituted benzylic residue, or an allylic residue; or R is represented by (X), where (X) is:

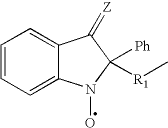

in which R₁ is:

C1-C18 methylenic alkyl residue and Z is defined as above;

and a substance acceptable from the dermatological or cosmetic point of view. This composition is suitable for the preparation of a dermatological or cosmetic composition for the treatment of skin, as recited in the attached claims.

Advantageously, Z is an NR₂ group, with R₂ a benzene group.

Advantageously, the Applicant has found the use of compounds with formula (I) useful for the preparation of a dermatological or cosmetic composition for the treatment of skin.

Preferably, the treatment is suitable for protecting skin from damage due to free radicals including those deriving from the action of solar radiation.

Preferably, the treatment is suitable for protecting skin from aging due to external agents.

Preferably, the treatment is suitable for protecting skin from certain pathologies such as: acne, cutaneous allergies due to external agents, eczema, dermatitis.

Advantageously, the composition, object of the present invention, contains a compound of formula (I) in an amount ranging from 0.1 to 5% in weight with respect to the total weight of the composition.

Preferably, the composition is in the form of a liquid, nebulized liquid, spray, gel or emulsion.

Preferably, the composition further contains one or more of the following: hydrophylic compounds, antimicrcbic agents, anti-inflammatory agents, vitamins, sugars, trace elements, enzymes and essential oils and organic and inorganic compounds with high UV light absorbing capacity.

With the aim of simplifying and making the description which follows as clear as possible, in the context of the present invention the following aromatic nitroxides will be abbreviated as follows:

Abbreviation MC4: mono-nitroxide of formula (I) in which R is an alkyl residue with C4, and Z is the NR₂ group, with R₂ being a phenyl group.

Abbreviation MC8: mono-nitroxide of formula (I) in which R is an alkyl residue with C8, and Z is the NR₂ group, with R₂ being a phenyl group.

Abbreviation DC4: bis-nitroxide of formula (I) in which R is represented by (X) where R1 is a methylenic C4 alkyl residue, and Z is the NR₂ group, with R₂ being a phenyl group.

Abbreviation DC8: bis-nitroxide of formula (I) in which R is represented by (X) where R1 is a methylenic C8 alkyl residue, and Z is the NR₂ group, with R₂ being a phenyl group.

The Applicant has selected certain nitroxide radicals as antioxidant agents following experimental tests. The antioxidant properties were studied in the laboratory in different biological systems. The systems employed are biological systems which mimic in vivo ones. The biological systems used are subjected to oxidative damage. Oxidative damage was induced in various ways. The Applicant carried out an experimental test to evaluate the peroxidation of unilamellar PC liposomes induced by the use of the Bu^{t}OOH/Cytochrome C system.

The unilamellar PC liposomes employed were prepared using the commonly accepted methods known by those skilled in this field. The unilamellar liposomes were prepared at a final phospholipid concentration of 11 mM. The liposomal dispersion was kept on ice and protected from sunlight. The peroxidation of these liposomes was monitored using a Gilson oxygraph and the data are reported in FIG. 1. The instrument was equipped with a Clark oxygen electrode. The measurements were conducted in a final volume of 1.8 ml and at a constant temperature of 37° C. After stabilization of the electrode, the consumption of oxygen in the suspension of unilamellar liposomes at a final concentration of 2.4 mM in phosphate buffer saline was induced by the addition of 25 μl of tert-butylhydroperoxide Bu^{t}OOH 7.3 M. Subsequently, 25 μl of a solution of Cytochrome C was added. The solution of Cytochrome C contained 1 mg of protein/ml.

After 80 seconds from the onset of peroxidation, the different nitroxide radicals at final concentration of 5 μM were added. The nitroxides were previously dissolved in acetonitrile (0.3% v/v). The reaction was monitored for 6 minutes. For each nitroxide tested, the extent of inhibition of the consumption of oxygen was calculated. The value of inhibition was calculated in the following way:

[Extent of oxygen consumed in 6 minutes in the presence of 5 μlM of acetonitrile (control sample, curve (g)) injected at point C (see FIG. 2)]−[Extent of oxygen consumed in 6 minutes in the presence of the nitroxides tested injected at point C]/[Extent of oxygen consumed in 6 minutes in the presence of 5 μl of acetonitrile (control sample curve (g)) injected at point C (see FIG. 2)]. The results are reported as a percentage of inhibition of the consumption of oxygen in FIG. 2.

The peroxidation of lipids of was studied by monitoring the consumption of oxygen. The peroxidation of unilamellar liposomes in the presence of various nitroxide compounds was investigated by measuring the amount of oxygen by means of a Gilson oxygraph.

The addition of tert-butylhydroperoxide Bu^{t}OOH followed by Cytochrome C induces a rpaid and constant consumption of oxygen which is suppressed to different extents after the addition of equimolar concentrations of the nitroxides tested as can be observed in FIG. 1.

Each individual recording is reported in FIG. 1, since the rate of oxygen consumption before the addition of the antioxidant compounds at point C is always constant. From these experiments, the percentage inhibition of oxygen consumption was calculated by subtraction as reported above. The results are shown in FIG. 2.

FIG. 2 shows that nitroxide DC4, at a concentration of 5 μM, is the best antioxidant agent in this reaction system. In fact, for the nitroxide radical DC4, the value of inhibition of oxygen consumption is almost equal to 80%. By increasing the concentration from 5 μM to 8 μM there is complete suppression, as observed in FIG. 2.

From FIG. 2, is can be deduced that the aliphatic nitroxide radical named TDN gives lower inhibition values with respect to DC4. Furthermore, the aliphatic nitroxide radical named TEMPO shows no protective effects against lipid peroxidation in this system and with the experimental conditions employed. Finally, always with reference to the nitroxide radical TEMPO, it was observed that in the same experimental conditions and at a higher concentration, no protective effects are detected.

The Applicant carried out an experimental test to evaluate the peroxidation of linolenic acid micelles induced using the Bu^{t}OOH/Cytochrome C system.

Similar experiments using the same experimental conditions were carried out on different lipid systems with respect to those reported in FIG. 2, consisting of linolenic acid micelles. The results are reported in FIG. 3. Linolenic acid micelles represent a more simple liquid system compared to unilamellar liposomes employed in the previous example. The linolenic acid micelles were prepared according to the commonly accepted methods known by the expert in this field. The linolenic acid micelles were prepared at a final concentration of 11 mM. The peroxidation of the micelles was followed by monitoring the consumption of oxygen in a volume of 1.8 ml at a constant temperature of 37° C. as described above.

The consumption of oxygen was induced in a micellar suspension at a final concentration of 3 mM in Tris-HClbuffer, pH 8.5 by addition of 14 μl of Bu^{t}OOH 7.3M. Subsequently, 14 μl of a solution of Cytochrome C (1 mg of protein/ml) were added. The peroxidation reaction was monitored in the presence and in the absence of 5 μM nitroxides, object of the present invention.

The percentage inhibition of oxygen consumption was evaluated in a similar way to that described above. The experimental values obtained are reported in FIG. 3.

The values reported in FIG. 3 show that bis-nitroxide radicals, object of the present invention such as those indicated with the abbreviations DC4 and DC8, show a percentage Inhibition of oxygen consumption in the micellar system which is double with respect to the value of the percentage of inhibition of oxygen consumption obtained from the mono-nitroxide radicals. However, both the mono-nitroxide compounds and bis-nitroxide compounds show a percentage inhibition of oxygen consumption superior to aliphatic nitroxide radicals commonly used such as TEMPO and TDN.

In this case, there is a clear difference between the protective effects of bis-nitroxides compared to their corresponding mono-nitroxide derivatives. All the bis-nitroxides inhibit approximately twice as much compared to the mono-nitroxides and furthermore, the protective effects of aromatic nitroxides, object of the present invention, are superior with respect to aliphatic nitroxides such as compounds named TDN and TEMPO.

In both experimental systems described above, the percentage of inhibition of oxygen consumption exerted by the compounds tested are dependent on their concentration.

The Applicant carried out an experimental test to evaluate the peroxidation of multilamellar PC liposomes, induced through the Bu^{t}OOH/Cytochrome C system.

The liposomes employed in this experimental system contain inside them the antioxidant agents. The antioxidants incorporated inside the multilamellar liposomal system before the onset of oxidation, show a greater protective effect with respect to when the antioxidant is not incorporated in the liposome but added subsequently. The multilamellar PC liposomes, with or without incorporation of the nitroxide radicals, were prepared adopting the same methodologies and with the same concentrations as described in the previous examples. The peroxidation of these liposomes was evaluated by monitoring the consumption of oxygen in a final volume of 1.8 ml and at a constant temperature of 37° C. in 5 mM of phosphate buffer saline, 0.1 mM EDTA, pH 7.4 as previously described.

The final concentration of the liposomes and of antioxidant agents, in the oxidation chamber, was 0.77 mM and 22 μM respectively. The oxidation was induced by addition of 30 Pl of Bu^{t}OOH 7.3 M followed by addition of 30 μl Cytochrome C (1 mg/ml of protein). The consumption of oxygen was monitored for 5 minutes. For each of the nitroxides tested, the degree of suppression of oxygen consumption was calculated. The value of inhibition was calculated in the following way: [Extent of oxygen consumed in 5 minutes in liposomes (without incorporation of antioxidant agents)]−[Extent of oxygen consumed in 5 minutes in liposomes in the presence of antioxidants incorporated inside them]/[Extent of oxygen consumed in 5 minutes in liposomes (without incorporation of antioxidant agents)]. The results are reported as a percentage of inhibition of oxygen consumption. The values are reported in FIG. 4 where it is possible to observe that the nitroxides, object of the present invention, incorporated inside the multilamellar liposomal system before the onset of the oxidation reaction, contribute to slowing down the oxidation reaction.

In the experimental test whose results are reported in FIG. 4, multilamellar liposomes which contain, incorporated inside them, the nitroxides object of the present invention and commonly known aliphatic nitroxides, were employed. In practice, the phospholipids are mixed with the nitroxide compounds before the formation of multilamellar liposomes. These kind of multilamellar liposomes were subjected to peroxidation by using the Bu^{t}OOH/Cytochrome C system. The consumption of oxygen was monitored as described above. The results reported in FIG. 6 show that incorporation of the aliphatic nitroxides TEMPO inside the liposomes had no effect, the aliphatic nitroxide TDN incorporated inside the liposomes inhibited peroxidation only by 45% while incorporation of the mono-nitroxide and bis-nitroxide compounds, object of the present invention, inhibited peroxidation approximately by 55-60%.

The Applicant carried out an experimental test to evaluate the oxidation of proteins induced by the azo-initiator, AAPH [2,2′-azobis(2-amidinopropane)dihydrochloride].

The protein samples were prepared by addition of 3 mg/ml of albumin in 50 mM phosphate buffer, 0.1 M EDTA, pH 7.4.

Subsequently, the protein samples were incubated at 50° C. for 60 minutes in the presence or absence of 5 mM of azo-initiator AAPH and/or 70 μM (final concentration) of nitroxide radical antioxidant agents, object of the present invention. The antioxidants were added to the protein as acetonitrile solution (2.5% v/v) at a final concentration as reported above. Subsequently, the azo-initiator compound AAPH was added.

The extent of protein oxidation was monitored by using the standard methods commonly known by the experts in this field such as the method of Levine et al. reported in the literature. The cited method employs the reaction of 2,4-dinitrophenylhydrazine (DNPH) with the carbonyl groups present in the oxidized protein. The results are reported in FIG. 5 in which the following is indicated on the x axis:

a. −AAPH: protein sample without azo-initiator;

b. +AAPH: protein sample containing azo-initiator,

c. protein sample containing mononitroxide compound of formula (I) in which R=C4 alkyl residue (MC4) and the azo-initiator AAPH;

d. protein sample containing bis-nitroxide compound of formula (I) in which R=(X) and (X) contains R1=C4 methylenic alkyl residue (DC4) and the azo-initiator AAPH;

e. protein sample containing the aliphatic nitroxide radical TEMPO and the azo-initiator AAPH;

f. protein sample containing the aliphatic nitroxide TDN and the azo-initiator AAPH.

With reference to the antioxidant activity evaluated for the new nitroxides in protein systems, it is important to underline the details of the results obtained. The results reported in FIG. 5 show that there is an increment in the carbonyl content of albumin after exposure to peroxide radicals produced by the azo-initiator, and that all the compounds, except for the aliphatic nitroxide TEMPO, reduce oxidative damage to different extents. The mono-nitroxide MC4 reduces protein damage approximately by 50%. The mono-nitroxice MC4 inhibits protein oxidation much better in comparison with both its corresponding bis-nitroxide and with the other compounds tested, for example TDN.

The aliphatic nitroxide TEMPO seems to actually increase protein oxidation induced by peroxyl radicals.

As reported above, the Applicant tested in the laboratory the nitroxides of general formula (I) in biological systems which have a similar behaviour to in vivo biological systems.

Another object of the present invention, is the method of preparation of compounds with formula (I). This method consists in the reaction of a suitable Grignard reagent with formula Y—Mg (—R) in which R represents a C 1-Cl 8 alkyl residue, a phenyl residue, a substituted phenyl group, a benzylic residue or substituted benzylic group or allylic residue; or formula Y—Mg—(—R—)—Mg—Y in which R represents a C1-C18 alkyl residue, with a substrate of the type:

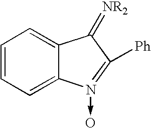

in a suitable solvent.

Advantageously, to prepare the compounds of formula (I) in which Z represents an atom of oxygen, the method, object of the present invention, foresees a further step of hydrolysis for obtaining the carbonyl group (C═O) in position 3.

The Applicant has found useful the employment of compounds of formula (I) as food preservative compounds. In particular, for foods such as fodder and seed oils.

Furthermore, the Applicant has found useful the employment of compounds of formula (I) as preservative compounds for paints, especially for paints based on siccative oils.

1. A dermatological or cosmetic composition comprising at least a nitroxide radical of general formula (I): 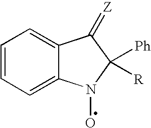
in which:
Z represents oxygen or an NR₂ group wherein R₂ comprises an alkyl, alkoxyl, aryl or phenyl group;
R represents an alkyl residue having between 1 and 18 carbon atoms, a phenyl residue or substituted benzene, a benzylic or substituted benzylic residue or alkyl residue; or R is represented by (X), where (X) is: 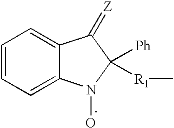
in which R1 is:
a methylenic alkyl residue having between 1 and 18 carbon atoms and Z is defined as above;
and a substance acceptable from the dermatological or cosmetic point of view.

2. A composition according to claim 1, wherein Z is the NR₂ group, wherein R₂ comprising a phenyl group.

3. A composition according to claim 1, wherein R is an alkyl residue having 4 carbon atoms.

4. A composition according to claim 1, wherein R is an alkyl residue having 8 carbon atoms.

5. A composition according to claim 1, wherein the compound of formula (I) has R=(X), where (X) is: 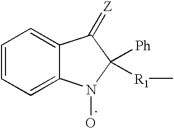
in which R1 is a C4 methylenic alkyl residue and Z is defined as above.

6. A composition according to claim 1, wherein the compound of formula (I) has a R=(X), where (X) is: 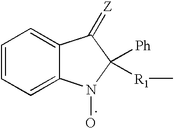
in which R1 is a C8 methylenic alkyl residue and Z is defined as above

7. A composition according to claims from 1, wherein the compound of formula (I) is present in an amount ranging from 0.1% to 5% in weight with respect to the total weight of the compound.

8. A composition according to claim 7, wherein the compound is in the form of: liquid, nebulized liquid, spray, gel or emulsion.

9. A composition according to claim 7, further contains at least one of the following: hydrophylic compounds, antimicrobic agents, anti-inflammatory agents, vitamins, sugars, trace elements, enzymes and essential oils and organic and inorganic compounds with high UV light absorbing capacity.

10. A method for the dermatological or cosmetic treatment of skin comprising applying onto the skin a composition in accordance with claim 8.

11. A method for the dermatological or cosmetic treatment of skin comprising applying onto the skin a composition in accordance with claim 9.

12. A method according to claim 11, wherein the treatment protects skin from damage induced by free radicals including those deriving from the action of solar radiation.

13. A method according to claim 11, wherein the treatment protects skin from aging due to external agents.

14. A method according to claim 11, wherein the treatment protects skin from certain pathologies such as: acne, cutaneous allergies due to external agents, eczema, or dermatitis.

15. A method according to claim 11, wherein the compound of formula (I) has Z of the NR₂ group with R₂ being a phenyl group.

16. A method according to claim 11, wherein the compound of formula (I) has R=C4 alkyl residue.

17. A method according to claim 11, wherein the compound of formula (I) has R=C8 alkyl residue.

18. A method according to claim 11, wherein the compound of formula (I) has R=(X), where (X) is: 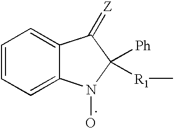
in which R1 is a C4 methylenic alkyl residue and Z is defined as above.

19. A method according to claim 11, wherein the compound of formula (I) has R=(X), where (X) is: 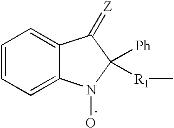
in which R1 is a C8 methylenic alkyl residue and Z is defined as above.

20. Use of compounds of formula (I) as claimed in claim 1, as a preservative for foods such as fodder and seed oils.

21. Use of compounds of formula (I) as claimed in claim 1, as a preservative compounds for paints, especially for paints based on siccative oils.

22. A method for the preparation of compounds of formula (I) as claimed in claim 1, the method consisting of the reaction between a suitable Grignard reagent with formula Y—Mg—(—R) in which R represents a C1-C18 alkyl residue, a phenyl residue or a substituted benzene residue, a benzylic or substituted benzylic residue or an allylic residue; or formula Y—Mg—(—R—)—Mg—Y in which R represents a C1-C18 alkyl residue, with a substrate of the type: 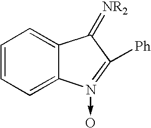
in a suitable solvent.

23. The method according to claim 22, characterized by the fact that to prepare the compounds with formula (I) in which Z represents an atom of oxygen, this method foresees a further hydrolysis step for obtaining the carbonyl group (C═O) in position 3.
